(19) United States
(12) Patent Application Publication
(10) Pub. No.: US 2007/0016190 A1
(43) Pub. Date: Jan. 18, 2007

(54) Dynamic spinal stabilization system

(75) Inventors: Jaime Martinez, Pompton Plains, NJ (US); Alexandre M. DiNello, Cotuit, MA (US)

Correspondence Address: GIBBONS, DEL DEO, DOLAN, GRIFFINGER & VECCHIONE, 1 RIVERFRONT PLAZA, NEWARK, NJ, 07102-5497, US

(73) Assignee: Medical Device Concepts LLC

(21) Appl. No.: 11/181,657

(22) Filed: Jul. 14, 2005

(51) Int. Cl. A61F 2/30

(52) U.S. Cl. 606/61

(57) ABSTRACT

A spinal stabilization system for installation to the posterior of the spinal column that includes a pair of anchoring members affixed to adjacent vertebrae and which may have screw threads to carry out that affixation. The anchoring members each have external head ends for the attachment of a flexible shaft that thus spans between the vertebrae and allows motion between those adjacent vertebrae. The flexible shaft is made of a monolithic body having no moving components that could generate debris and fail mechanically. Exemplary flexible shafts suitable for use in this system includes one having at least one slot that may be a spiral slot, a shaft having pairs of oppositely disposed slots formed in the body with adjacent pairs angularly displaced apart or a flexible shaft having a serpentine slot formed therein. The flexible shaft is controlled to have a desired flexibility.

### FIELD OF THE INVENTION

The present invention relates to a system for interconnecting vertebrae of a spinal column of a patient, and, more particularly, to a system that is affixed to the vertebrae and which has a predetermined dynamic flexibility to allow motion between the vertebrae while providing support for the spinal column.

### BACKGROUND OF THE INVENTION

In the field of spinal devices and techniques, there is a common practice today of fusing adjacent vertebrae together in order to compensate for a damaged disc located intermediate those vertebrae. Unfortunately, the use of the fusion technique reduces the flexibility of the spinal column and results in some loss of activity of the patient. With the fusion procedure, there may be emplaced both an anterior spacer and rigid posterior element that “lock” the adjacent vertebrae together with the appropriate spacing, thus eliminating pain and restoring the correct anatomical position.

With the advent, however of motion devices, such as disc replacement devices, within the anterior vertebrae disc space, there is a need for a dynamic system for the posterior segments as well as the prior anterior affixation devices.

Accordingly, it would be advantageous to have a posterior dynamic spinal stabilization system that could be installed to the posterior of the spinal column and allow a range of motion to that posterior of the spinal column that restores the natural biomechanics of the spinal column to allow the natural range of motions of the spinal column.

### SUMMARY OF THE INVENTION

Therefore, in accordance with the present invention there is a posterior dynamic spinal stabilization system that is intended, for example, for the thoracic, cervical or lumbar sections of the spinal column and which provides a positive, yet flexible means of stabilizing the posterior of the spinal column. The stabilization system of the present invention can, therefore, allow the spinal column to regain its natural motion, that is, the present system can allow the spinal column to move with ranges of natural motion such as, preferably, movement in rotation to range from about greater than 0 to about 30 degrees, in medial/lateral motion in the range from about greater than 0 to about 45 degrees and for anterior/posterior (flexion/extension) in the range from about greater than 0 to about 120 degrees and, more preferably, movement in medial/lateral motion in the range from about greater than 0 to about 5 degrees and for anterior/posterior (flexion/extension) in the range from about greater than 0 to about 12 degrees. The present stabilization system can be used where there is a natural or artificial disc intermediate adjacent vertebrae, where there is a fusion cage or even where a spacer, including a multi-axial spacer, is utilized.

The stabilization system includes at least two anchoring members that are adapted to be affixed proximate to the posterior of the vertebrae of the spinal column and, normally, there are two of such anchoring members that are affixed to each adjacent vertebrae, that is, there are a pair of anchoring members affixed to each of the adjacent vertebrae.

While the method of affixing the anchoring members may vary, in the exemplary embodiment described herein, the anchoring members are specially constructed screws having screw threads that are screwed into the adjacent vertebrae to become firmly affixed to the vertebrae and each of the screws has an external head end that extends outwardly from the screw thread and thus projects outwardly from the posterior side of the vertebrae.

A flexible shaft is joined to the external head ends of the anchoring members such that, in one embodiment illustrated, there are a pair of such flexible shafts, each affixed to one of the pair of anchoring members affixed to the adjacent vertebrae. The flexible shafts are, therefore, oriented generally parallel to each other along the posterior of the spinal column spanning between the adjacent vertebrae. As will be seen, although the exemplary embodiment described herein relates to the affixing of the stabilization system between two adjacent vertebrae, it can be seen that the stabilization system can be utilized with two or more vertebrae, that is, the present inventive system can be used to span three, four or more vertebrae.

The flexible shafts are specially constructed to be strong, monolithic devices comprising a body having one or more slots formed therein in order to provide the necessary flexibility to the shafts, and, of course, to the adjacent vertebrae. For example, there may be single spiral slot or plurality of successive spiral slots formed in the body in the manner as described in U.S. Pat. No. 5,488,761 of Leone, the disclosure of which is incorporated herein in its entirety by reference. As an alternate flexible shaft, the flexible shaft may comprise a body having alternating pairs of oppositely disposed slots formed in the body with alternating pairs of slots being angularly offset, for example, at an angle of about 90 degrees as shown and described in co-pending patent application of Jaime Martinez, entitled “Flexible Shaft” and filed Jun. 3, 2005 as Ser. No. ______, the disclosure of which is hereby incorporated herein by reference in its entirety. As a still further alternative, the flexible shaft may be constructed in accordance with the helix-like slot forming the flexible member of U.S. Pat. No. 6,053,922 of Krause et al, and the disclosure of that patent is also incorporated herein in its entirety by reference.

The aforedescribed flexible shafts have the added advantage in that the degree of flexibility can be designed into the particular flexible shaft, that is, the flexibility of the shaft can be designed so as to be predetermined by selecting among a number of parameters, such as but not limited to changing the spacing of the slots, selecting the material for making the flexible shaft or changing the cross section of the shaft and any one or more of those selections can be made to design into the flexible shaft, the flexibility that is desired in the ultimate stabilization system. Accordingly, the amount of flexibility of the stabilization system can be designed in accordance with the needs of the particular spinal column. In addition, with a monolithic body, the flexible shaft has no moving components that could generate debris or fail mechanically.

Not only can the flexibility of the flexible shaft be predetermined to a desired flexure as a uniform movement, but due to the manufacturing methods of the aforedescribed flexible shafts, the amount or degree of stiffness or flexure of the shaft may vary depending upon the direction of that flexing, that is, the flexibility of the flexible shaft may be different depending on the direction of the flexing of the shaft. As such, the flexibility of the flexible shaft may allow movement of the patient side to side having different flexibility than the front to back movement and the like, so that the degree of flexibility of the spinal column can be customized in accordance with the desire of the physician in returning the patient to the normal natural motion of the spinal column.

The flexible shafts, using the aforedescribed slots, can have cylindrical bodies or, more preferable, can be of other cross sectional configurations such as oval, oval with flattened opposed surfaces, rectangular or other shapes that allow the flexing of the flexible shafts by means of the slot or slots formed therein and yet be readily and conveniently attachable to the external head ends of the anchoring members.

The affixation of the flexible shaft to the external head ends of the anchoring members can be carried out by a variety of methods and devices. As an example the external head ends may be specially dimensioned so as to pass through holes in the flexible shafts such that a ring can be forced onto the distal ends of the external head ends in a force fit relationship with the flexible shaft sandwiched therebetween to be held in position to the anchoring members. Alternatively, the rings could be affixed by swaging, set screws or other means.

Another means of affixing the flexible shaft to the anchoring members is to provide a receiver in the external head end of the anchoring members that receives the ends of the flexible shafts and locks the flexible shaft to the anchoring members by means such as set screws threaded into corresponding threads formed in the receivers. While the aforedscribed examples are illustrative, there are, of course, other and differing means of affixing the flexible shafts to the anchoring members that could be employed to carry out that affixation without departing from the spirit and intent of the present invention.

The posterior spinal stabilization system is also installed by means of a novel method. In particular, the anchoring members are initially affixed to the vertebrae of the spinal column, preferably using a pair of anchoring members to be affixed to each of two adjacent vertebrae. The anchoring members are preferable screws having threads and external head ends that extend outwardly from the vertebrae after the anchoring members have been fully screwed into the vertebrae in a tight, secured fashion. At the external head ends, there can be receivers so that the flexible shaft is affixed to the receivers.

In attaching the flexible shaft to the external head ends of the anchoring members, the flexible shaft can be pre-bent to a desired bend orientation. The flexible shaft can be pre-bent to achieve varying degrees of lordosis (backward curvature) or kyphosis (forward curvature) prior to being affixed to the anchoring members and also the curvature of the flexible member depends upon the location along the spinal column, i.e. the cervical region would have a kyphotic curve while the lumbar region would have a lordotic curve. Thus, once installed to the vertebrae, the flexible shaft will provide the proper, desired curvature for the spinal column.

The actual placement of the flexible shaft in making up the dynamic stabilization system may also be by differing means. For example, the procedure can be minimally invasive such as by installing the flexible shaft by means of a guide wire through one or more small incisions in the patient. That guide wire itself can be shaped into the preferred curvature, that is, the flexible shaft can be slid over a lordotic configured guide wire as the flexible shaft passes over the guide wire. Alternatively, if the present dynamic stabilization system is installed during major surgery to install, for example, a replacement disc, the patient is already fully accessible for installation of the dynamic stabilization system.

Thus, in the method, the flexible shaft is affixed to the anchoring members so that the flexible shaft can provide a good support to the spinal column to allow movement of the patient's spinal column. In many cases, if the stabilization system is installed by major surgery with a substantial incision, the flexible shaft may well be pre-bent into the desired configuration, whereas if the flexible shaft is inserted with a minimal incision, the use of a guide wire may be preferred where the guide wire is bent into the desired curvature and the that the flexible shaft takes on that curvature as it slides over the guide wire.

Other features of the posterior dynamic spinal stabilization system of the present invention and its method of installation will become more apparent in light of the following detailed description of a preferred embodiment thereof and as illustrated in the accompanying drawings.

### BRIEF DESCRIPTION OF THE DRAWINGS

FIG. 1 is a partially exploded view illustrating the affixation of the posterior dynamic spinal stabilization system of the present invention to adjacent vertebrae of a spinal column;

FIG. 2 is a posterior view of the system of FIG. 1 attached to the spinal column;

FIG. 3 is a side view of an alternative embodiment of the present spinal stabilization system of the present invention affixed to a spinal column;

FIG. 4 is a perspective view of the stabilization system of FIG. 3;

FIG. 5 is a side view of an exemplary flexible shaft that is usable with the present invention;

FIG. 6 is a side view of another exemplary shaft that is usable with the present invention;

FIG. 7 is a side view of a still further exemplary shaft that is usable with the present invention;

FIG. 8 is a side view of a exemplary pre-bent flexible shaft that is usable with the present invention;

FIG. 9 is an exploded view illustrating the method of installing the present spinal stabilization system to the spinal column of a patient; and

FIG. 10 is a view of the guide structure used in installing the spinal stabilizing system of the present invention to a patient.

### DETAILED DESCRIPTION OF THE INVENTION

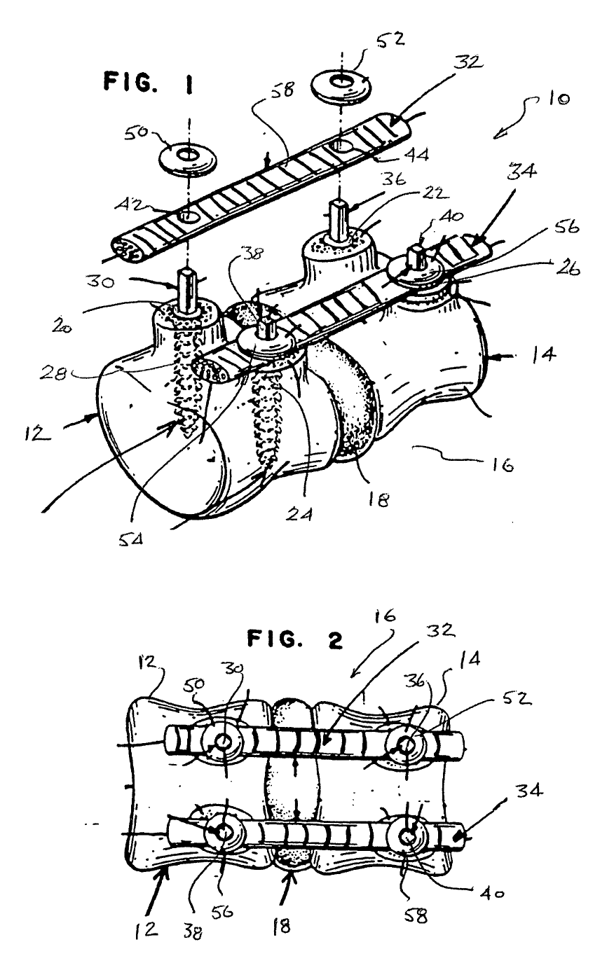

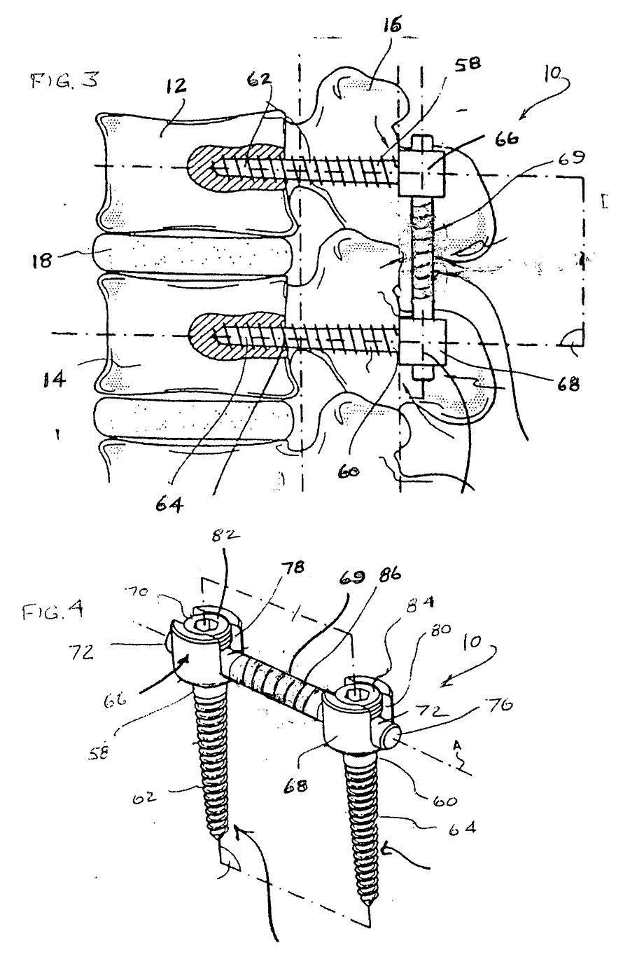

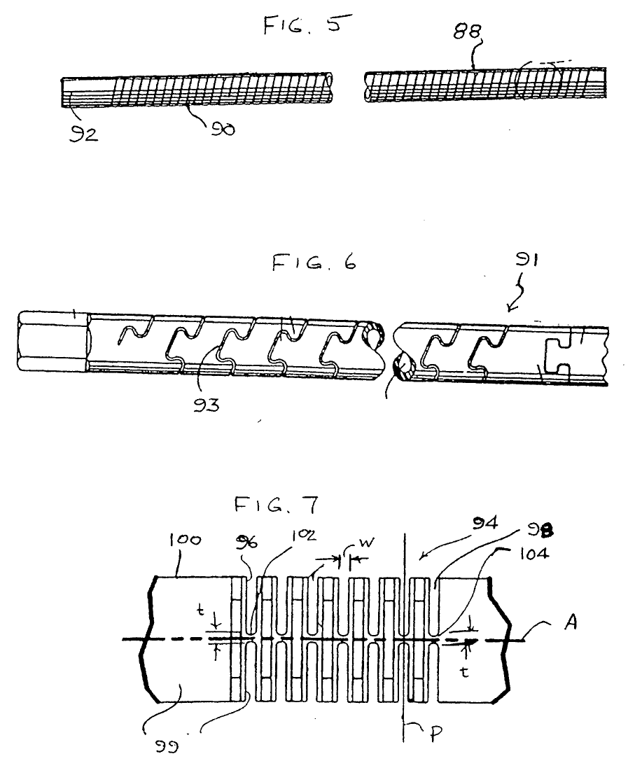

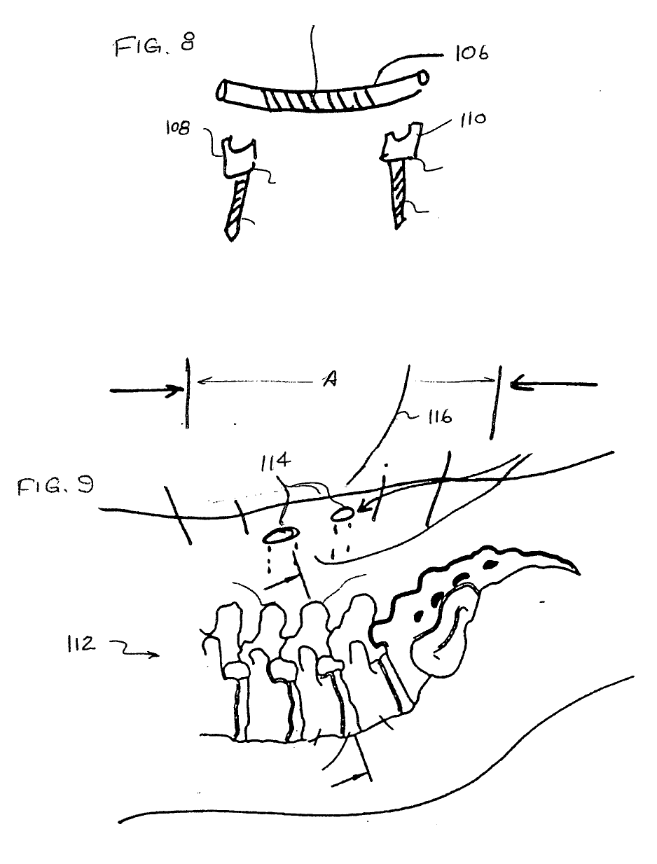

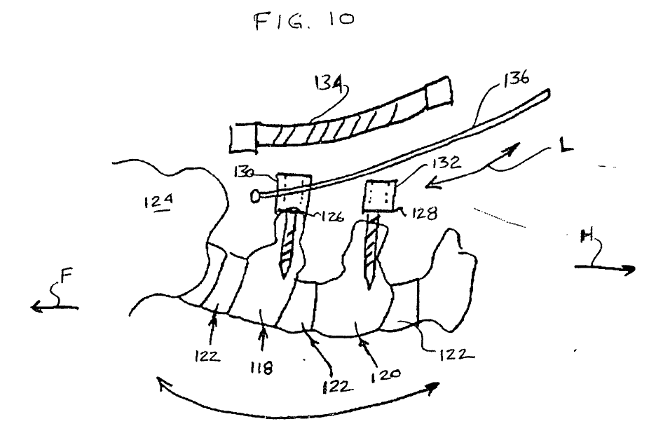

Referring now to FIG. 1, there is shown a partially exploded view illustrating an exemplary dynamic spinal stabilization system 10 of the present invention affixed to the adjacent vertebrae 12, 14 of spinal column 16. As can be seen, the vertebrae 12, 14 are separated by a disc 18 that may be a natural disc or may be a prosthetic device that has taken the place of a normal disc by a replacement thereof. As shown, the upper portion of the vertebrae 12, 14 is the posterior side facing the posterior of the patient and the lower portion is the anterior side facing inwardly of the patient. Therefore it can be seen that the stabilization system is affixed proximate to the posterior of the spinal column 16.

There are a plurality of anchoring members 20, 22, 24 and 26 and are components of the present stabilization system 10 and two of the anchoring members, 20 and 24 are positioned side by side affixed to vertebra 12 and anchoring members 22, 26 are positioned side to side affixed to vertebra 14. Since all of the anchoring members are identical, only anchoring member 20 will be described as typical and anchoring member 20 includes threads 28 that are screwed into the vertebra 12 in order to solidly affix the anchoring member 20 to the vertebra 12.

The anchoring member 20 has an external head end 30 extending from the threads 28 and consequently also extending outwardly from the vertebra 12. As shown, the external head end 30 is square in cross section, however other cross sectional configurations could also be employed, included a threaded external head end 30.

A pair of flexible shafts 32, 34 are affixed to the external head end 30 of the anchoring member 20 as well as to the external head ends 36, 38, 40 of the other anchoring members 22, 24, and 26 respectively. The flexible shaft 32 has a pair of holes 42, 44 formed therein that are spaced apart a predetermined distance so as to receive the external head ends 30, 36 in mounting the flexible shaft 32 to the anchoring members 20, 22. In the same manner, flexible shaft 34 can be placed over the external head ends 38, 40 that pass through corresponding holes in the flexible shaft 34 in order to mount the flexible shaft 34 to the anchoring members 24, 26 to join the adjacent vertebrae 12, 14.

Finally, the flexible shafts 32, 34 are affixed to the respective anchoring members 20, 22 and 24, 26 by means of securing devices that are affixed to the external head ends 30, 36, 38 and 40 and one such securing device can be rings 50, 52, 54 and 56 that fit over the respective anchoring members 20, 22, 24, and 26 in a tight fit to secure the flexible shafts 32, 34 to the anchoring members 20, 22, 24 and 26. Other securing devices could also be used, for example, nuts could be used if the external head ends of the anchoring members are threaded shafts.

In any instance, the use of a slotted flexible shafts 32, 34 allows the flexible shafts to be designed for the desired flexibility by changing the configuration of the slot, the material of the body, the cross section of the body as well as other design changes so that the designer can have the proper flexibility of the flexible shafts 32, 34 depending upon the desired characteristics of the spinal column to which the flexible shafts 32, 34 are being installed.

It should be noted that with the FIG. 1 embodiment where two parallel flexible shafts 32, 34 are employed, the use of the Leone spiral slotted shaft may provide more directional flexibility and be capable of flexing both back and forth as well as laterally whereas the use of the oppositely disposed slots for the flexible shaft may limit the lateral motion of the flexible shaft and, therefore, also limit the side to side motion of the spinal column.

Turning briefly to FIG. 2, there is shown a rear or posterior view of the spinal column 16 and illustrating the flexible shafts 32, 34 installed to the posterior side of the adjacent vertebrae, 12, 14 to provide a dynamic stabilization to those vertebrae. Thus, as can be seen, both of the flexible shafts 32, 34 span across the adjacent vertebrae 12, 14 so as to provide support thereto, and yet, due to the flexible nature of the flexible shafts 32, 34, there is a dynamic movement between the vertebrae in order to allow the spinal column 16 natural movement as the person carries out normal motions.

Turning now to FIG. 3, there is shown a side view of an alternative embodiment of the present posterior dynamic spinal stabilization system 10. As can be seen, again there are adjacent vertebrae 12, 14 with a disc 18 located intermediate thereto and the stabilization system is affixed to the posterior side of the spinal column 16. In this embodiment, there are two anchoring members 58, 60 illustrated joining the vertebrae 12, 14, however, as with the prior embodiment, there may be four anchoring members with two flexible shafts employed.

In any event, the anchoring members 58, 60 have threads 62, 64 and external head ends 66, 68. A flexible shaft 69 spans between and is affixed to both of the external head ends 66, 68. The span of the flexibility of the stabilization system 10 is illustrated by the length dimension D so that flexing is allowed between the vertebrae 12, 14.

Turning now to FIG. 4, taken along with FIG. 3, there is a perspective view of the stabilization system 10 of FIG. 3 isolated from the various vertebrae. Thus, in FIG. 4, the anchoring members 58, 60 are shown with the screw threads 62, 64 and with the external head ends 66, 68. In this embodiment the external head ends 66, 68 have receivers 70, 72 formed therein and into which the opposed flexible shaft ends 74, 76 are affixed. The receivers 70, 72 have internal threads and transverse slots 78, 80 that cross the internal threads so that the opposed flexible shaft ends 74, 76 can be nested within the transverse slots 78, 80 and set screws 82, 84 are screwed into the internal threads within the receivers 70, 72 to affix the flexible shaft 69 to the opposed flexible shaft ends 74, 76.

The flexible shaft 69 again is constructed with one or more slots 86 to achieve the desired flexibility and, again, the flexibility can be built into the design of the flexible shaft 69 as desired for the particular patient.

Turning now to FIG. 5, there is shown a side view of an exemplary flexible shaft 88 that can be used with the present invention. In FIG. 5, the flexible shaft 88 is constructed in accordance with U.S. Pat. No. 5,488,761 of Leone and generally comprises a helical slot 90 that is formed into a shaft 92 and may have slot interruptions. The flexible shaft provides some rotating or torsional give when rotary motion is along the flexible shaft 88 so that the flexible shaft 88 can have both flexibility along its longitudinal axis but also a small degree of rotational motion is allowed along that longitudinal axis.

In FIG. 6, there is shown a side view of a further exemplary flexible shaft 91 that can be used with the present invention and where there is a specially formed serpentine slot 93 along the length of the flexible shaft 91 that is constructed in accordance with the disclosure of Krause et al U.S. Pat. No. 6,053,922. The spiral shaft can be cut into the surface of the flexible shaft 91 by means of continuously rotating the shaft while providing relative motion of a cutting piece along the longitudinal length of the shaft. Thus, to adjust the pitch of the helical slots, the speed of the rotation of the shaft can be adjusted with respect to the relative longitudinal movement of the cutting tool or piece. The cutting step can further include inserting a cannulated tube into the hollow rod and forcing a pressurized fluid through the cannulated tube against an inner surface of the hollow rods and through the slots to clean that shaft.

Next, in FIG. 7, there is shown a side view of a still further exemplary flexible shaft 94 that can be used with the present invention. In this embodiment, there are a plurality of pairs of oppositely disposed slots 96 formed in a tubular body 99 and, as shown, those slots 96 are specially located and configured so as to create the desirable features of the present flexible shaft. The slots 96 are each comprised of an elongated opening 98 that is located along the peripheral outer surface 100 of the tubular body 99 and extend inwardly toward the longitudinal axis A of the flexible shaft 94. The elongated openings 98 of each pair of oppositely disposed slots 96 are located in a common plane, illustrated as P in FIG. 7, that is, at a right angle or 90 degrees to the longitudinal axis A with the elongated openings 98 of each pair formed in the same plane orthogonal to the longitudinal axis A. As can be seen in FIG. 7, the pairs of slots 96 are illustrated to extend inwardly such that each slot of a pair of slots 96 lies along the same plane P as the elongated openings 98, however, the slots 96 may be angled with respect to that plane or tapered inwardly such that while the elongated openings 98 of each pair of slots may be along the same lateral plane, the slots 96 themselves may be directed inwardly at an angle with respect to that plane.

The slots 96 are formed in the peripheral outer surface 100 of the tubular body 98 such that each slot 96 is less than 180 degrees about the peripheral outer surface 100 of the tubular body 98. Accordingly, since the pairs of slots 96 each are grouped in oppositely disposed slots 96, each slot is cut into the tubular body 99 and the slots 96 approach each other but terminate at ends 102 short of reaching the center of the tubular body 99, that is, the pairs of slots 96 are non-continuous and do not reach the longitudinal axis A as shown in FIG. 7.

Therefore, between each of the ends 102 of a pair of slots 96 there are formed web sections 104 that separate the ends 102 of the pairs of slots 96. Thus, each pair of oppositely disposed slots 96 as illustrated in FIG. 7 are in a common plane with the web sections 104 separating the ends 102 of each pair of slots that are formed in the tubular body 99 to approach each other but fall short of reaching the midpoint or longitudinal axis A of the tubular body 99. As such, the web sections 104 carry the rotational movement along the flexible shaft 94 while maintaining torque along that flexible shaft 94.

The pairs of slots 96 are alternately angularly oriented with respect to each other around the outer peripheral surface of the tubular body 99, that is, each succeeding pair of oppositely disposed slots 96 is rotated or displaced a predetermined angular amount from the orientation of the succeeding pair of slots 96. In the embodiment shown in FIG. 7, that displacement or rotation is about 90 degrees such that the slots 96 are formed in the tubular body every quarter of a turn. As such, there are at least a first and second pair of oppositely disposed slots 96 formed in the tubular body 99 with, for example, the first pair having one orientation and the next or second pair of slots 96 oriented 90 degrees rotated with respect to the first pair of slots 96 and so on throughout the tubular body 99.

While the angular displacement is illustrated in FIG. 7 to be 90 degrees, other angular displacements may be utilized and that angular displacement need not be the same or even consistent between successive pairs of slots 96.

The width w of the slots 96 can be predetermined in accordance with the desired flexibility of the completed flexible shaft 94, that is, the larger the width dimension w, the more flexible the eventual flexible shaft 94. The same is true of the depth of the slots 96 as the oppositely disposed slots approach each other nearing the midpoint or longitudinal axis A of the tubular body 99 i.e. the smaller the thickness t of the web sections 104 between the slots of each pair, the more flexible the flexible shaft 94 becomes. In one suitable embodiment, the thickness t of the web sections 104 is about the same, dimensionally, as the width w of the slots 96.

As can therefore be seen, the flexibility of the flexible shaft 94 can be different depending on the particular direction of flexing of the flexible shaft 94. One means of accomplishing that different flexibility would be to establish differing widths of pairs of slots 96 along two opposite sides of the flexible shaft 94 such that the flexibility in one direction of the pairs of slots 96 is different than the flexibility in another direction of motion, such as a direction at 90 degrees to the first direction. As such, the present flexible shaft 94 can be affixed to the vertebrae of the patient in a particular orientation where the front to back flexibility of the spinal column can be different, and possibly more flexible, than the flexibility of the spinal column in a side to side direction. As such, in one embodiment, the flexible shaft is designed and constructed so as to have a range of motion in rotation in the range of from about greater than 0 to about 30 degrees, a medial/lateral motion in the range of from about greater than 0 to about 45 degrees and an anterior/posterior (flexion/extension) in the range from about greater than 0 to about 120 degrees and that range of motion can be readily built in to at least one of the shafts herein disclosed. More preferably, movement in medial/lateral motion can be in the range from about greater than 0 to about 5 degrees and for anterior/posterior (flexion/extension) in the range from about greater than 0 to about 12 degrees.

The formation of the slots in this and other flexible shafts can be accomplished by a variety of methods including milling the slots into the tubular body, using wire electrical discharge machining, water-jet machining, laser machining, spark erosion machining or rotary cutting machining. The material for the flexible shafts can be any hard, rigid material including, but not limited to stainless steel, titanium, chrome cobalt molybdenum, polymers and carbon fiber composites.

Accordingly, any of the flexible shafts illustrated in FIGS. 5-7 can be used with the present invention and can be designed to have the desired flexibility to support the vertebrae while allowing dynamic motion therebetween. Also, it should be noted that the flexible shaft can have differing degrees of flexibility depending on the direction of flexure, that is, as described, the flexible shafts may have a differing amount of flexibility for forward and rearward motion as opposed to side to side motion. In addition, the shafts may have the flexibility vary along the longitudinal axis of the shafts, that is, certain linear areas of a shaft may have a differing flexibility than other linear areas of the same shaft so that the physician can select and use a customized shaft depending upon the condition of the patient and the particular use of the stabilization device.

Turning now to FIG. 8, there is shown a schematic view of a pre-bent flexible shaft 106. The flexible shaft 106 can thereof be affixed to the external head ends 108, 110 in a curved disposition such as shown in FIG. 8 illustrating a lordotic bend in the flexible shaft 106 as an example. That curvature can be predetermined so as to be preformed or the curvature can be created during the procedure to a desired curvature by the physician installing the flexible shaft 106 for the particular patient. The lordotic bend is illustrated, however, the bend can be any preferred bend by the physician, including a kyphotic curve. As illustrated, the flexible shaft 106 is shown with the spiral slot embodiment of FIG. 5, however, the particular flexible shaft 106 can readily be of the type illustrated in FIGS. 6 and 7.

Turning now to FIG. 9, there is shown an exploded view of a spinal column 112 in order to illustrate exemplary methods of installing the dynamic stabilization system of the present invention. Again, the dynamic stabilization system of the present invention can be installed to the posterior of the spinal column 112 in the lumbar, cervical or thoracic regions. The surgical procedure can be carried out by means of open surgery where the surgery entails access to the spinal column 112 along the area designated generally as A such that the surgeon has full access to the spinal column of the patient and can install the various components of the stabilization system. A less invasive surgery can be where the access is more restricted than along the area A or the surgical technique could be a minimally invasive procedure where a plurality of through portals 114 are made in the patient for insertion and installation of the stabilization system. In the event of a minimal invasive procedure, the flexible shaft 108 (FIG. 8) can be inserted by means of a guide wire 116 and the flexible shaft 106 slid over that guide wire to the proper position to be affixed to the spinal column 112. As stated, the guide wire 116 can itself be curved to the appropriate curve desired for the flexible shaft so that the flexible shaft can take on the curvature of the guide wire 116 as it is being inserted into the patient. Alternatively, of course, as explained with respect to FIG. 8, the flexible shaft may be pre-bent into the desired curvature.

Turning finally to FIG. 10, there is shown a schematic view illustrating a procedure for installing the spinal stabilization system of the present invention to the spinal column of a patient. As can be seen in FIG. 10, there are adjacent vertebrae 118, 120 separated by a disc 122. The arrow H indicates the direction of the head of the patient while, correspondingly, the arrow F indicates the direction of the feet of the patient. The sacrum 124 is also illustrated, however, the present spinal stabilization system can, as explained, be used with adjacent vertebrae of the spinal column along the thoracic, cervical or lumbar regions.

As also can be seen, there are anchoring members 126 and 128 that have been affixed to the adjacent vertebrae 118, 120 by being screwed into those vertebrae 118, 120 leaving the external head ends 130, 132, respectively, extending outwardly from the vertebrae 118, 120. The anchoring members 126, 128 can be constructed as shown and described with respect to FIGS. 3 and 4 or may be constructed in alternative embodiments.

In any event, in order to install the flexible shaft 134 to the external head ends 130, 132 of the anchoring members 126, 128, a guide wire 136 is inserted through the anchoring members 126, 128 and the flexible shaft 134 slid over the guide wire 136 so as to be positioned within the external head ends 126, 128 and secured in place therein as described in the prior illustrated mechanisms. Upon affixing the flexible shaft 134 to the anchoring members, 126, 128, the guide wire 136 can, of course, be removed.

In the illustration of FIG. 10, the guide wire 136 can be bent to the particular curvature desired for the flexible shaft 134 so that the flexible shaft 134 ultimately takes on the curvature of the guide wire 136. Accordingly, as explained, the curvature can be a lordotic curve as noted by the arrow L, or, alternatively the curvature may be, for example, a kyphotic curve and can therefore be any alternative curvature desired by the physician to suit the needs of the patient.

The previously described guide wire or guide structure can be used where the flexible shaft is hollow or has a lumen extending fully along the longitudinal axis thereof. In the event a solid flexible shaft is utilized, an alternative guide structure can be employed, such as a guide structure that at least partially surrounds the flexible shaft. An example would be a trough or semi-cylindrical tube into which the flexible shaft can pass such that the flexible shaft would, as with the guide wire, take on the curvature of the guide structure as desired by the physician.

While the present invention has been set forth in terms of a specific embodiment or embodiments, it will be understood that the bracket system herein disclosed may be modified or altered by those skilled in the art to other configurations. Accordingly, the invention is to be broadly construed and limited only by the scope and spirit of the claims appended hereto.

We claim:

1. A stabilization system for a spinal column comprising:
at least two anchoring members each anchoring member adapted to be affixed to adjacent vertebrae of a spinal column,
a flexible shaft for joining the at least two anchoring members so as to enable desired movement of the vertebrae of a patient's spinal column by allowing relative movement between the at least two anchoring members, the flexible shaft having a plurality of pairs of non-continuous slots oppositely disposed comprising elongated openings formed in the outer peripheral surface in a common plane, said slots extending inwardly from the elongated openings toward but not reaching the longitudinal axis of the shaft to form web sections between the slots of each pair of slots, and each succeeding pair of slots being spaced apart along the longitudinal axis and being angularly displaced at an angular displacement from a preceding pair of slots.

2. The stabilization system as defined in claim 1 wherein said shaft is a tubular body.

3. The stabilization system as defined in claim 2 where the common plane is orthogonal to the longitudinal axis of the tubular body.

4. The stabilization system as defined in claim 1 where the angular displacement between successive pairs of slots is about 90 degrees.

5. The stabilization system as defined in claim 1 wherein the pairs of slots have external ends and wherein the web sections are formed separating the external ends of each of the slots of a pair of slots.

6. The stabilization system as defined in claim 1 wherein the slots extend linearly along a portion of the flexible shaft.

7. The stabilization system as defined in claim 1 wherein each pair of slots is formed in the common plane orthogonal to the longitudinal axis of the shaft.

8. The stabilization system as defined in claim 1 wherein at least one slot tapers inwardly in the direction toward the longitudinal axis of the shaft.

9. The stabilization system as defined in claim 1 wherein the anchoring members have screw threads that are screwed into the posterior of adjacent vertebrae.

10. The stabilization system as defined in claim 9 wherein the at least two anchoring members have external head ends extending outwardly from the screw threads to allow the flexible shaft to be affixed to the external head ends of the at least two anchoring members.

11. The stabilization system as defined in claim 10 wherein the external head ends have affixation devices adapted to fit over the external head ends to retain the flexible shaft to the at least two anchoring members.

12. The stabilization system as defined in claim 11 wherein the affixation devices comprise rings having internal holes adapted to tightly fit over the external head ends to secure the rings to the at least two anchoring members.

13. The stabilization system as defined in claim 10 wherein the external head ends have internally threaded receivers adapted to receive set screws to affix the flexible shaft to the external head ends of the at least two anchoring members.

14. A method for making a spinal stabilization system comprising the steps of:
providing a hollow rod having a longitudinal axis;
cutting one or more spiral slits during rotation of said rod to form a flexible shaft;
providing at least two anchoring members for securing to corresponding vertebrae of a patient; and
attaching the flexible shaft to the at least two anchoring members so as to enable movement of the flexible shaft in relation to a patient's spinal column.

15. The method according to claim 14, wherein said step of cutting comprises adjusting the speed of rotation with respect to axial movement of said hollow rod during the cutting to control the pitch of said helical slots.

16. The method according to claim 14, wherein said hollow rod is made from a material selected from the group consisting of stainless steel, titanium, chrome cobalt molybdenum, polymers and a carbon fiber composite.

17. The method according to claim 16, wherein said step of cutting comprises one of wire electrical discharge machining, water-jet machining, laser machining, milling, spark erosion machining and rotary cutting machining.

18. The method according to claim 14, wherein the cutting step further comprises the steps of:
providing a cannulated tube for inserting into said hollow rod; and
forcing a pressurized fluid through said cannulated tube against an inner surface of said hollow rod and through said slots to clean said shaft.

19. A method for making a spinal stabilization system comprising the steps of:
providing a hollow rod having a longitudinal axis;
cutting one or more slots along a serpentine helical path in the hollow rod to form a flexible shaft, said plurality of slots having a substantial length and width extending within a region around the hollow rod;
providing at least two anchoring members for securing to corresponding vertebrae of a patient; and
attaching the flexible shaft to the at least two anchoring members so as to enable movement of the flexible shaft in relation to a patient's spinal column.

20. A method for making a spinal stabilization system comprising the steps of:
providing a hollow rod having a longitudinal axis;
cutting one or more slots along a serpentine helical path in the hollow rod to form a flexible shaft, said plurality of slots having a substantial length and width extending within a region around the hollow rod;
providing at least two anchoring members for securing to corresponding vertebrae of a patient; and
attaching the flexible shaft to the at least two anchoring members so as to enable movement of the flexible shaft in relation to a patient's spinal column.

21. A stabilization system for a spinal column comprising:
at least two anchoring members each anchoring member adapted to be affixed to adjacent vertebrae of a spinal column,
a flexible shaft for joining the at least two anchoring members so as to enable desired movement of the vertebrae of a patient's spinal column by allowing certain relative movement between the at least two anchoring members within the range of between about greater than 0 to about 45 degrees flexion medial/lateral and within the range of between about greater than 0 to about 120 degrees flexion anterior/posterior.

22. The stabilization system of claim 21 wherein the flexible shaft allows relative movement between the at least two anchoring members within the range of between about greater than 0 to about 5 degrees flexion medial/lateral and within the range of between about greater than 0 to about 12 degrees flexion anterior/posterior.

23. The stabilization system of claim 21 wherein the flexible shaft has a plurality of pairs of non-continuous slots oppositely disposed comprising elongated openings formed in the outer peripheral surface in a common plane, said slots extending inwardly from the elongated openings toward but not reaching the longitudinal axis of the shaft to form web sections between the slots of each pair of slots, and each succeeding pair of slots being spaced apart along the longitudinal axis and being angularly displaced at an angular displacement from a preceding pair of slots to provide said certain relative movement.

24. The stabilization system of claim 21 wherein the flexible shaft has at least one serpentine helical-like slot formed therein along its linear length.

25. The stabilization system of claim 21 wherein the flexible shaft has at least one spiral slot formed therein along its linear length.

26. The stabilization system of claim 21 wherein the flexible shaft allows rotational movement between the at least two anchoring members in the range of from about 1 to about 30 degrees.

27. A stabilization system for a spinal column comprising:
at least two anchoring members each anchoring member adapted to be affixed to adjacent vertebrae of a spinal column,
a flexible shaft for joining the at least two anchoring members so as to enable desired movement of the vertebrae of a patient's spinal column by allowing certain relative movement between the at least two anchoring members in the medial/lateral direction and in the anterior/posterior direction, said flexible shaft having a different degree of flexibility for movement in the medial/lateral direction than the anterior/posterior direction.

28. A method for installing a spinal stabilization system comprising the steps of:
securing at least two anchoring members to corresponding vertebrae of a patient, each of said at least two anchoring members including an external head end thereof;
aligning a flexible shaft to the at least two anchoring members; and
attaching the flexible shaft to the at least two anchoring members to enable movement of the flexible shaft in relation to the spinal column.

29. The method for installing a spinal stabilization system as defined in claim 28 wherein the step of securing at least two anchoring members comprises screwing an anchoring member into each of the vertebrae.

30. The method for installing a spinal stabilization system as defined in claim 28 wherein the step of attaching the flexible shaft to the at least two anchoring members comprises initially bending the flexible shaft to a desired curvature before affixing the flexible shaft to the at least two anchoring members.

31. The method for installing a spinal stabilization system as defined in claim 28 wherein the step of providing a flexible shaft comprises providing a flexible shaft having at least one spiral slot formed therein along its linear length.

32. The method for installing a spinal stabilization system as defined in claim 28 wherein the step of providing a flexible shaft comprises providing a flexible shaft having at least two pairs of oppositely disposed slots formed therein with a pair of slots being angularly displaced with respect to an adjacent pair of slots.

33. The method for installing a spinal stabilization system as defined in claim 32 wherein each adjacent pair of slots is displaced 90 degrees.

34. The method for installing a spinal stabilization system as defined in claim 28 wherein the step of providing a flexible shaft comprises providing a flexible shaft having at least one serpentine helical-like slot formed therein along its linear length.

35. A method for installing a spinal stabilization system comprising the steps of:
securing at least two anchoring members to corresponding vertebrae of a patient, each of said at least two anchoring members including an external head end thereof;
bending a flexible shaft into a desired bent orientation; and
aligning the bent flexible shaft to the at least two anchoring members; and
attaching the flexible shaft to the at least two anchoring members to enable movement of the flexible shaft in relation to the spinal column.

36. The method for installing a spinal stabilization system as defined in claim 35 wherein said desired bent orientation corresponds to lordosis for installation in the lumbar region of the spine.

37. The method for installing a spinal stabilization system as defined in claim 35 wherein said desired bent orientation corresponds to kyphosis of the spine.

38. The method for installing a spinal stabilization system as defined in claim 35 wherein the step of bending a flexible shaft comprises providing a guide wire having a predetermined bend and sliding the flexible shaft over the guide wire to cause the flexible shaft to acquire the same bend as the guide wire.

39. A method for making a spinal stabilization system comprising the steps of:
providing a tubular body having a longitudinal axis, an external peripheral surface and external ends, forming a plurality of pairs of oppositely disposed slots in the body to create a flexible shaft, the slots having elongated openings formed in the peripheral surface of the tubular body in a common plane orthogonal to the longitudinal axis of the tubular body and extending inwardly therefrom toward but not reaching the longitudinal axis of the tubular body to form web sections therebetween, alternately spacing every other pair of slots to be at an angular displacement with respect to the preceding pair of slots;
providing at least two anchoring members for securing to corresponding vertebrae of a patient, each of said anchoring members including an external head end thereof; and
attaching the flexible shaft to the at least two anchoring members so as to enable movement of the flexible shaft in relation to a patient's spinal column.

40. The method of claim 39 wherein the step of alternately spacing the pairs of slots comprises spacing the pairs of slots at an angular displacement of about 90 degrees.

41. The method of claim 39 wherein the step of forming first and second pairs of slots comprises the step of using electrical discharge machining.

42. The method of claim 39 wherein the step of forming the pairs of oppositely disposed slots comprises milling the slots into the tubular body.

43. The method of claim 39 wherein the step of forming the pairs of oppositely disposed slots comprises forming the pairs of slots at differing distances between successive pairs of slots along the longitudinal axis of the tubular body.

44. The method of claim 39 wherein the step of forming the pairs of oppositely disposed slots comprises forming pairs of slots having differing depths extending inwardly toward the longitudinal axis.
